(19) United States
(12) Patent Application Publication
(10) Pub. No.: US 2006/0229667 A1
(43) Pub. Date: Oct. 12, 2006

(54) Device forming epilating forceps

(76) Inventor: Herve Hoppenot, Paris (FR)

Correspondence Address: EPSTEIN DRANGEL BAZERMAN & JAMES, LLP, 60 EAST 42ND STREET, SUITE 820, NEW YORK, NY, 10165, US

(21) Appl. No.: 10/554,468

(22) PCT Filed: Apr. 23, 2004
(86) PCT No.: PCT/FR04/01004

§ 371 Date: Mar. 10, 2006

(30) Foreign Application Priority Data
Apr. 24, 2003 (FR) 03/05075

(51) Int. Cl. A61B 17/50

(52) U.S. Cl. 606/210

(57) ABSTRACT

The device (10) comprises a stationary body (12), a pincer member (14) comprising first and second pincer branches (14A, 14B) mounted to move in the stationary body (12) between a rest position and a cocked position, return means (16) for resiliently returning the pincer member (14) to the rest position, and locking means (18) for locking the pincer member (14) in the cocked position against the return force of the pincer member (14), and releasable by means of a trigger member (20) that can be activated by a user. The device (10) further comprises blocking means (32) for blocking the pincer branches (14A, 14B) in a pinched-together position, which blocking means are active during a displacement stroke of the pincer member (14) between its cocked position and its rest position, without the user needing to maintain any action on the trigger member (20).

The present invention relates to a device forming tweezers.

In the prior art, a tweezer-forming device is already known of the type comprising:

a stationary body;

a pincer member comprising first and second pincer branches mounted to move in the stationary body between a rest position and a cocked position;

return means for resiliently returning the pincer member to the rest position; and

locking means for locking the pincer member in the cocked position against the return force of the pincer member, and releasable by means of a trigger member that can be activated by a user.

A device of that type is described in U.S. Pat. No. 4,240,435, for example.

The pincer member of the tweezer-forming device described in U.S. Pat. No. 4,240,435 comprises two pincer branches each forming a lever of the third kind.

It is recalled that a lever is a simple machine that is generally categorized in three kinds.

A lever is said to be of the first kind, or a middle-fulcrum lever, when the hinge axis of the lever (fulcrum) is placed between firstly a drive portion of the lever on which a force for driving the lever (or power) is applied, and secondly an active portion of the lever on which a resisting force (or load) acts.

A lever is said to be of the second kind, or a middle-load lever, when the active portion of the lever is placed between the hinge axis and the drive portion of the lever.

Finally, a lever is said to be of the third kind, or a middle-power lever, when the drive portion of the lever is placed between the hinge axis and the active portion of the lever.

In U.S. Pat. No. 4,240,435, the branches of the pincer member can be moved from a spaced-apart position towards which they are returned resiliently, and a pinched-together position.

When the pincer member is in the cocked position, its branches are in the spaced-apart position.

A tweezer-forming device of the above-specified type is particularly suited to plucking undesirable hairs from a human being, in particular hairs situated in sensitive locations such as the ears, the nostrils, or the eyebrows.

That tweezer-forming device is usually presented to a hair for removal with the pincer member being in the cocked position in which the pincer branches are spaced apart.

Actuating the trigger member has the effect of unlocking the pincer member which is then returned resiliently towards its rest position. During this return movement, the pincer member takes hold of the hair and plucks it out.

The return movement of the pincer member to the rest position is very quick, so the hair is plucked in a manner that is practically painless.

In the device described in U.S. Pat. No. 4,240,435, the trigger member comprises two opposing trigger pushers mounted to move relative to the stationary body. Each trigger pusher co-operates with one of the pincer branches of the pincer member. Those two trigger pushers are provided with ball bearings facilitating relative movement between the branches of the pincer member and the trigger pushers.

The two trigger pushers serve firstly to unlock the pincer member in the cocked position and to hold the two branches of the pincer member in the pinched-together position during the return stroke of the pincer member towards its rest position, so as to pluck out the hair.

It should be observed that the structure of those trigger pushers, including ball bearings, is relatively complex.

An object of the invention is to propose a tweezer-forming device of the above-specified type that is simple in structure and inexpensive to manufacture.

To this end, the invention provides a tweezer-forming device of the above-specified type, characterized in that it includes blocking means for blocking the pincer branches in a pinched-together position, which blocking means are active during a displacement stroke of the pincer member between its cocked position and its rest position, without the user needing to maintain any action on the trigger member.

By means of the invention, the branches of the pincer member are held pinched together during the return stroke of the pincer member towards its rest position in such a manner as to pluck out a hair, automatically and without the user needing to maintain any action on the trigger member.

Furthermore, since the trigger member does not serve to hold the branches of the pincer member pinched together, its structure can be relatively simple.

According to other characteristics of the tweezer-forming device:

the blocking means comprise two substantially-parallel slider surfaces formed in the body, between which surfaces the pincer member is arranged so that each branch of the pincer member co-operates with a corresponding slider surface during the displacement stroke of the pincer member between its cocked position and its rest position, the spacing between the slider surfaces being small enough to hold the branches pinched together;

each of the two branches of the pincer member forms a lever of the third kind, the branches being displaceable between a spaced-apart position and a pinched-together position, the branches preferably being returned resiliently towards the spaced-apart position;

the branches of the pincer member are in the spaced-apart position while said pincer member is in the cocked position;

the device includes a slider mounted on the body to be displaceable between a rest position and a cocked position, in such a manner as to be accessible at least in part from outside the body, and coupling means for coupling the slider with the pincer member in such a manner as to drive the pincer member between its rest position and its cocked position;

the coupling means comprise complementary drive abutments secured to the slider and to the pincer member;

the device includes return means for resiliently returning the slider to the rest position, which means are preferably distinct from the return means for returning the pincer member to the rest position;

the body is of generally elongate, tubular shape, the slider being carried by a proximal end of the body and being substantially coaxial with said body;

the body is generally of elongate tubular shape, the slider being carried by a side wall of the body and being slidable between proximal and distal ends of the body;

the trigger member is generally in the form of a pushbutton that is movable transversely relative to the body;

the trigger member is generally in the form of a lever hinged to the body about an axis that extends substantially transversely relative to the body;

the locking means comprise complementary locking abutments secured to the body and to the pincer member;

the locking abutment of the pincer member is defined by a shoulder formed on its first branch forming the end of a ramp for retracting the locking abutment of the pincer member; and

the trigger member is suitable for co-operating with the retraction ramp.

The invention can be better understood on reading the following description given purely by way of example and made with reference to the accompanying drawings, in which:

FIGS. 1 to 4 are axial section views of a tweezer-forming device in a first embodiment of the invention, showing four successive configurations in the operation of the device; and

FIGS. 5 to 8 are views analogous to FIGS. 1 to 4 respectively, showing a tweezer-forming device in a second embodiment of the invention.

FIGS. 1 to 4 show a tweezer-forming device in a first embodiment of the invention given overall reference 10.

This device 10 comprises a stationary body 12 of generally elongate, tubular shape. The section of the body 12 may be substantially square or circular, for example.

The device 10 also comprises a pincer member 14 comprising first and second pincer branches 14A and 14B.

The pincer member 14 is mounted to move in the body 12 between a rest position as shown in FIG. 1, and a cocked position, as shown in particular in FIG. 3.

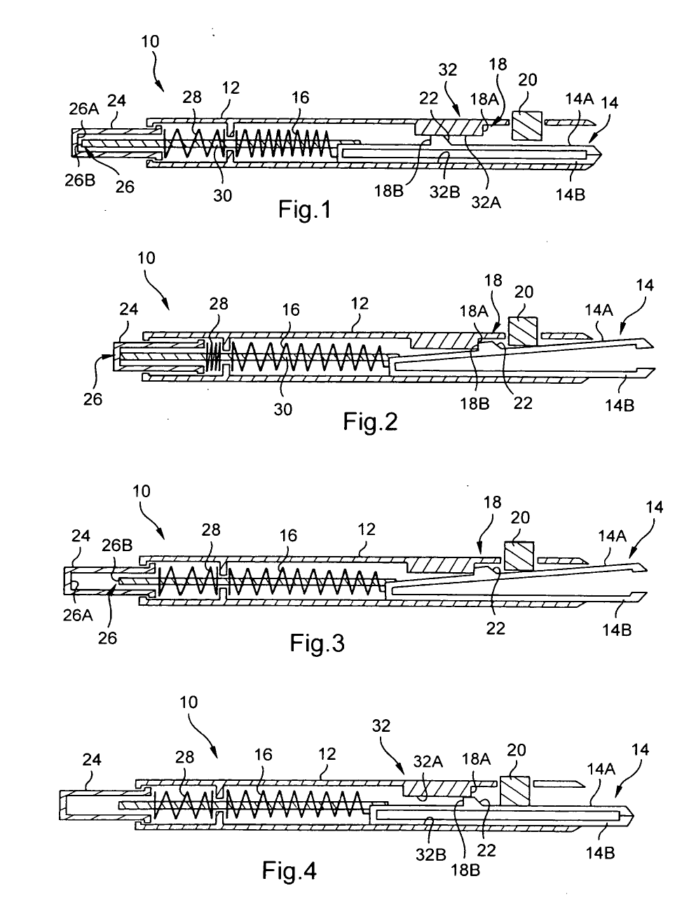

The first and second branches 14A and 14B of the pincer member 14 form respective levers of the third kind and can be moved between a pinched-together position as shown in FIGS. 1 and 4 and a spaced-apart position, as shown in FIGS. 2 and 3. The first and second branches 14A and 14B are preferably returned resiliently towards the spaced-apart position.

It should be observed that the pincer branches 14A and 14B are in the spaced-apart position when the pincer member 14 in the cocked position (see FIGS. 2 and 3).

Each of the pincer branches 14A and 14B is provided with a free end. These free ends serve to pinch onto a hair when the pincer branches 14A and 14B are in the pinched-together position.

The device 10 also comprises a traction spring 16 forming resilient return means for returning the pincer member 14 to its rest position.

The device 10 further comprises locking means 18 for locking the pincer member 14 in the cocked position. These locking means 18 serve to oppose the return force of the spring 16 while the pincer member 14 is in the cocked position (see FIGS. 2 and 3).

The locking means 18 can be released with the help of a trigger member 20 suitable for being activated by a user. In the first embodiment of the invention, the trigger member 20 is generally in the form of a pushbutton mounted on the body 12 to move transversely relative thereto.

The locking means 18 comprise complementary locking abutments 18A and 18B secured respectively to the body 12 and to the pincer member 14.

More particularly, it should be observed that the locking abutment 18B of the pincer member 14 is defined by a shoulder formed on the first branch 14A of the pincer member 14. This shoulder defines the end of a projection forming a ramp 22 for retracting the locking abutment 18B of the pincer member 14.

It should be observed that since the first branch 14A of the pincer member forms a lever of the third kind, the locking abutment 18B of the pincer member 14 and the retraction ramp 22 are placed on the drive portion of this lever of the third kind.

To move the pincer member 14 between its rest and cocked positions, the device 10 includes a slider 24 that is movable relative to the body 12 between a rest position as shown in particular in FIG. 1 and a cocked position as shown in FIG. 2.

It should be observed that the slider 24 is accessible, at least in part, from outside the body 12.

The slider 24 is coupled with the pincer member 14 by coupling means 26 so as to drive the pincer member 14 between its rest and cocked positions.

The slider 24 is returned resiliently to the rest position by a compression spring 28 forming resilient return means that are distinct from the return means of the pincer member 14 as formed by the traction spring 16.

It should be observed that the traction spring 16 (for returning the pincer member 14) and the compression spring 28 (for returning the slider 24) lie substantially on the same axis.

The means for coupling the slider 24 to the pincer member 14 comprise complementary drive abutments 26A and 26B secured respectively to the slider 24 and to the pincer member 14.

In the first embodiment of the invention, the slider 24 is generally in the form of a cap, and it is carried at a proximal end of the body 12. Under such circumstances, it should be observed that the slider 24 is substantially coaxial with the body 12.

The coupling abutment 26A secured to the slider 24 is formed inside a proximal end of the slider 24. The coupling abutment 26B secured to the pincer member 14 is formed on a proximal end of a member 30 that axially extends the pincer member 14 away from the free ends of its pincer branches 14A and 14B.

In order to pluck a hair during resilient return of the pincer member 14 towards its rest position, the device includes blocking means 32 for blocking the pincer branches 14A, 14B in their pinched-together position. These blocking means 32 are active in particular during a displacement stroke of the pincer member 14 from its cocked position as shown in FIG. 2 or 3 towards its rest position as shown in FIG. 1.

The blocking means 32 preferably comprise two substantially parallel slider surfaces 32A, 32B, formed inside the body 12.

It should be observed that the pincer member 14 is arranged between these slider surfaces 32A and 32B in such a manner that each branch 14A, 14B of the pincer member 14 co-operates with a corresponding slider surface 32A, 32B during the displacement stroke of the pincer member from its cocked position towards its rest position.

The spacing between the slider surfaces 32A and 32B is small enough to keep the branches 14A and 14B pinched together while taking advantage of the relative resilience of these branches in a direction extending transversely to the pincer member 14.

The main aspects of the operation of the tweezer-forming device 10 that are associated with the invention are described below.

Initially, the device 10 is in a rest configuration, as shown in FIG. 1, in which the pincer member 14 is in its rest position (retracted into the body 12).

The user then actuates the slider 24, pushing it from its rest position towards its cocked position, as shown in FIG. 2. This has the effect of driving the pincer member 14 by co-operation between the complementary drive abutments 26A and 26B from its rest position towards its cocked position, as shown in FIG. 2.

When user action on the slider 24 is interrupted, the slider is urged towards its rest position by the compression spring 28, as shown in FIG. 3.

It should be observed that while the pincer member 14 is being moved from its rest position towards its cocked position, the ramp 22 of the first branch 14A co-operates with a corresponding slider surface 32A, such that the branches 14A and 14B of the pincer member 14 are pinched together. Nevertheless, once the pincer member 14 has reached its cocked position, the ramp 22 escapes from the slider surface 32A such that the branches 14A and 14B of the pincer member are returned resiliently to the spaced-apart position, as shown in FIGS. 2 and 3.

When the pincer member 14 is in the cocked position, the locking abutments 18A and 18B co-operate with each other so as to oppose the resilient return force delivered by the traction spring 16.

With the device 10 in the configuration shown in FIG. 3, the user places the free ends of the branches 14A and 14B of the pincer member on either side of a hair that is to be plucked.

Thereafter, the user actuates the trigger member 20, thereby pinching together the branches 14A and 14B of the pincer member and releasing the locking means 18. The pincer member 14 is then automatically returned towards its rest position by the traction spring 16. During the return stroke of the pincer member 14 towards its rest position, the two slider surfaces 32A and 32B co-operate respectively with the first branch 14A of the pincer member 14 via the ramp 22, and with the second branch 14B of the pincer member 14. This has the effect of blocking the pincer branches 14A and 14B in a pinched-together position while the pincer member 14 is moving from its cocked position, as shown in FIG. 3 to its rest position, as shown in FIG. 1, and passing via the intermediate position shown in FIG. 4.

It can thus be observed that the slider surfaces 32A and 32B enable the pincer branches 14A and 14B to be held in the pinched-together position while the pincer member 14 is being returned towards its rest position, without there being any need for the user to maintain any action on the trigger member 20.

Since the action of the trigger member 20 is to pinch a hair between the free ends of the branches 14A and 14B of the pincer member, the return movement of the pincer member 14 towards its rest position causes the hair to be plucked out quickly and painlessly.

FIGS. 5 to 8 show a tweezer-forming device 10 in a second embodiment of the invention.

In these figures, elements that are analogous to elements of the above figures are designated by references that are identical.

In this embodiment, the slider 24 is carried by a side wall 12L of the body. The slider 24 is arranged on the body 12 between the proximal and distal ends of the body 12.

Furthermore, unlike the first embodiment of the invention, in the second embodiment of the invention, the trigger member 20 is generally in the form of a lever hinged on a distal end of the body 12 about an axis X extending substantially transversely relative to the body 12.

The trigger member 20 is preferably arranged in line with the path followed by the slider 24.

More particularly, it should be observed that the trigger member 20 forms a lever of the second kind, with the active portion of the lever serving to co-operate with the retraction ramp 22.

In the second embodiment of the invention, both of the return springs 16 and 28 are arranged inside the body 12, substantially in parallel with each other. Furthermore, the spring 28 for returning the slider 24 acts in traction and not in compression as in the first embodiment of the invention.

It should also be observed that in the second embodiment of the invention, the drive abutment 26B is arranged on a member that extends the second branch 14B of the pincer member 14 transversely.

The tweezer-forming device 10 of the second embodiment of the invention operates in analogous manner mutatis mutandis to the device of the first embodiment of the invention.

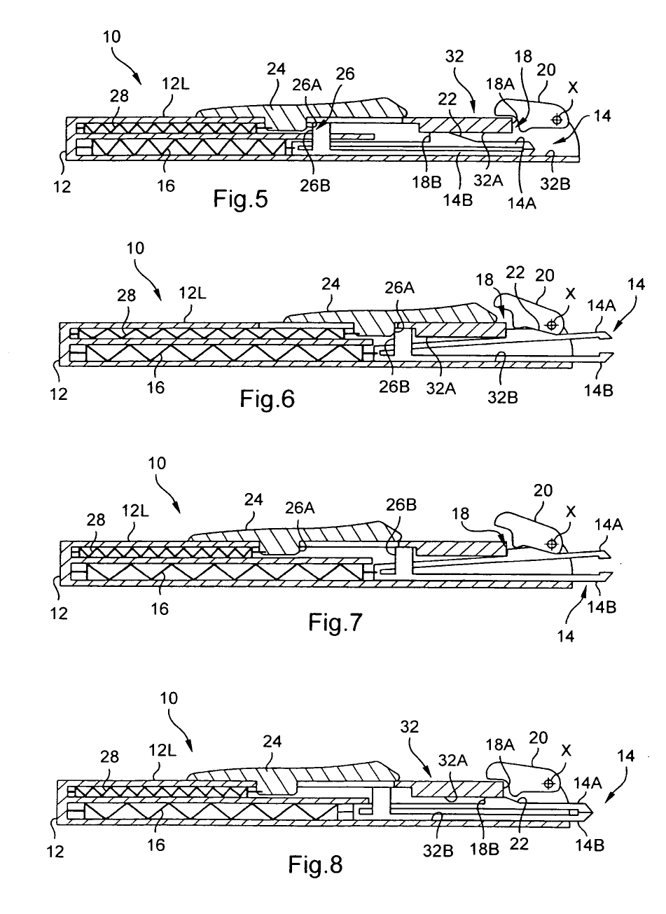

Nevertheless, it should be observed that the device 10 of the second embodiment of the invention is more ergonomic to handle than is the device of the first embodiment of the invention, in particular concerning the displacement of the slider 24 from its rest position, as shown in FIG. 5, towards its cocked position, as shown in FIG. 6.

Since the trigger member 20 in the second embodiment of the invention is arranged longitudinally in line with the slider 24, the user can release the locking means 18 of the pincer member 14 by acting on the lever-forming trigger member 20 with the same digit as is used to move the slider 24, e.g. the thumb, without needing to change significantly the position of the device 10 in the hand.

It should be observed that the slider 24 is returned towards its rest position as soon as the user releases it, as shown in FIG. 7, so as to avoid impeding the user while actuating the trigger member 20, and so as to avoid constituting an obstacle to the stroke of the pincer member 14 towards its rest position once the locking means 18 has been released.

Naturally, the tweezer-forming device 10 of the invention could, like conventional tweezers, be used for functions other than plucking out a hair, for example it could be used for extracting a foreign body from the skin, such as a splinter.

1. A tweezer-forming device (10) of the type comprising:
a stationary body (12);
a pincer member (14) comprising first and second pincer branches (14A, 14B) mounted to move in the stationary body (12) between a rest position and a cocked position;
return means (16) for resiliently returning the pincer member (14) to the rest position; and
locking means (18) for locking the pincer member (14) in the cocked position against the return force of the pincer member (14), and releasable by means of a trigger member (20) that can be activated by a user;
the device (10) being characterized in that it includes blocking means (32) for blocking the pincer branches (14A, 14B) in a pinched-together position, which blocking means are active during a displacement stroke of the pincer member (14) between its cocked position and its rest position, without the user needing to maintain any action on the trigger member (20).

2. A device (10) according to claim 1, characterized in that the blocking means (32) comprise two substantially-parallel slider surfaces (32A, 32B) formed in the body (12), between which surfaces the pincer member (14) is arranged so that each branch (14A, 14B) of the pincer member co-operates with a corresponding slider surface (32A, 32B) during the displacement stroke of the pincer member (14) between its cocked position and its rest position, the spacing between the slider surfaces (32A, 32B) being small enough to hold the branches (14A, 14B) pinched together.

3. A device (10) according to claim 1 or claim 2, characterized in that each of the two branches (14A, 14B) of the pincer member (14) forms a lever of the third kind, the branches being displaceable between a spaced-apart position and a pinched-together position, the branches (14A, 14B) preferably being returned resiliently towards the spaced-apart position.

4. A device (10) according to claim 3, characterized in that the branches (14A, 14B) of the pincer member (14) are in the spaced-apart position while said pincer member (14) is in the cocked position.

5. A device (10) according to any preceding claim, characterized in that it includes a slider (24) mounted on the body (12) to be displaceable between a rest position and a cocked position, in such a manner as to be accessible at least in part from outside the body (12), and coupling means (26) for coupling the slider with the pincer member (14) in such a manner as to drive the pincer member (14) between its rest position and its cocked position.

6. A device (10) according to claim 5, characterized in that the coupling means (26) comprise complementary drive abutments (26A, 26B) secured to the slider (24) and to the pincer member (14).

7. A device (10) according to claim 5 or claim 6, characterized in that it includes return means (28) for resiliently returning the slider (24) to the rest position, which means are preferably distinct from the return means (16) for returning the pincer member (14) to the rest position.

8. A device (10) according to any one of claims 5 to 7, characterized in that the body (12) is of generally elongate, tubular shape, the slider (24) being carried by a proximal end of the body (12) and being substantially coaxial with said body (12).

9. A device (10) according to any one of claims 5 to 7, characterized in that the body (12) is generally of elongate tubular shape, the slider (24) being carried by a side wall (12L) of the body (12) and being slidable between proximal and distal ends of the body (12).

10. A device (10) according to claim 8 or claim 9, characterized in that the trigger member (20) is generally in the form of a pushbutton that is movable transversely relative to the body (12).

11. A device (10) according to claim 8 or claim 9, characterized in that the trigger member (20) is generally in the form of a lever hinged to the body (12) about an axis (X) that extends substantially transversely relative to the body (12).

12. A device (10) according to any preceding claim, characterized in that the locking means (18) comprise complementary locking abutments (18A, 18B) secured to the body (12) and to the pincer member (14).

13. A device (10) according to claim 12, characterized in that the locking abutment (18B) of the pincer member (14) is defined by a shoulder formed on its first branch (14A) forming the end of a ramp (22) for retracting the locking abutment (18B) of the pincer member (14).

14. A device (10) according to claims 11 and 13 taken together, the device being characterized in that the trigger member (20) is suitable for co-operating with the retraction ramp (22).
